(19) United States
(12) Patent Application Publication
(10) Pub. No.: US 2008/0161743 A1
(43) Pub. Date: Jul. 3, 2008

(54) Ablation device having a piezoelectric pump

(76) Inventor: John E. Crowe, Menlo Park, CA (US)

Correspondence Address: SJM/AFD-WILEY, 14901 DEVEAU PLACE, MINNETONKA, MN, 55345-2126, US

(21) Appl. No.: 11/646,527

(22) Filed: Dec. 28, 2006

(51) Int. Cl. A61B 18/14

(52) U.S. Cl. 604/20

(57) ABSTRACT

An ablating device having one or more piezoelectric micropumps for delivering a flowable material to a target tissue. The device may be coupled to a controller configured to monitor tissue temperature, ablation element temperature, transducer impedance and/or degree of contact between the ablation elements and a tissue and to adjust the flow rate of the flowable material in response to changes in temperature, transducer impedance and/or degree of contact between the ablation elements and the tissue. Methods of ablating tissue using devices of the present invention are also described.

### BACKGROUND OF THE INVENTION

a. Field of the Invention

The instant invention generally relates to medical devices incorporating a piezoelectric micropump and methods for using the devices for treatments that benefit from fluid perfusion or cooling fluids. In particularly preferred embodiments, the instant invention relates to devices and methods for treating electrophysiological diseases of the heart, such as epicardial ablation for the treatment of atrial fibrillation. More generally, the present invention relates to devices and methods for the improved delivery of a flowable material to a target tissue.

b. Background Art

In performing a number of minimally invasive procedures, such as cardiac ablation of an epicardial surface, a flowable material, such as saline, can be used for the dual purposes of cooling tissue and conducting energy from the ablation element to the tissue. Typically, the source of flowable material is an intravenous bag of saline in which the flow is gravity driven and may be coupled to the ablation device with a standard luer connection. During an ablation procedure, for example, the tissue may be maintained at an appropriate temperature to avoid burning. The tissue temperature is monitored throughout the procedure, and the flow rate of the flowable material, for example saline, is manually increased by manipulation of a valve coupled to the gravity-driven saline bag when the tissue temperature exceeds a threshold level. As the flowable material cools the tissue, the flow rate can be manually decreased.

A flowable material, for example saline, may also be used to conduct energy from an ablation element to the tissue and to ensure adequate contact between the ablation element and the tissue. The degree of contact between the ablation element and the tissue is monitored throughout the procedure, and the flow rate of the flowable material is manually adjusted by manipulation of a valve coupled to the gravity-driven bag to ensure appropriate contact.

A disadvantage of the existing devices and procedures is that they require manual adjustment of the flow rate of the flowable material throughout the ablation procedure to maintain an appropriate tissue temperature and sufficient contact between the ablation device and the tissue. A valve coupled to the gravity-driven bag is manually opened and closed to regulate the flow rate, and adjustments must be made throughout the procedure to ensure proper flow. What are needed, therefore, are devices and methods for improved delivery of a flowable material, such as saline, to the tissue.

### BRIEF SUMMARY OF THE INVENTION

The present invention meets these and other objectives by providing devices and methods for ablating tissue using an irrigation fluid flowing from the controlled vibration of a piezoelectric element or pump. In general aspects, the invention comprises a medical device that incorporates a piezoelectric pump for delivering a biocompatible fluid into the body during a surgical procedure. In a preferred embodiment, the medical device is a cardiac tissue-ablating device comprising one or more ablating elements, where the ablating elements benefit from the use of cooling fluids or functional fluids, such as physiological saline solutions. A variety of ablating elements, designs or systems using ablating elements, and other devices using irrigation fluid are known in the art and can be adopted for use according to the invention.

In a preferred embodiment, the ablating device is one employing a high energy cell or element or multiple high energy cells or elements. One or more piezoelectric micropumps can be used to irrigate the tissue being ablated by each cell, especially in conjunction with a passageway to convey a flowable material of fluid between a piezoelectric micropump and a location proximate to an ablation element.

The type or design of the piezoelectric micropump selected for use can preferably be controlled by the desired flow rate for the ablating cell or element used. For example, in the case of an ablating device using at least one high intensity focused ultrasound cell or element, the flow rate to the cell is about 1 cc/sec or more. The size of the micropump fluid chamber, the construction of the membrane and piezoelectric material or disk for causing the fluid flow, as well as valves and other design options for the micropump can all influence the range of flow rates possible from the vibrational frequency range for a selected piezoelectric material, power source, or pump.

Thus, in one aspect the invention encompasses an irrigated medical device comprising at least one high intensity focused ultrasound cell, and at least one micropump, where the micropump includes a piezoelectric material or disk contacting a fluid chamber though a membrane or diaphragm, and a conduit to permit the flow of a fluid into and out of the chamber. As described below, the micropump can be provided as a small element capable of being used inside the body during a surgical procedure.

In another aspect, the invention encompasses a system for ablating cardiac surface tissue or atrial tissue comprising an ablation device with at least one ablation element, at least one piezoelectric micropump and a passageway to convey a flowable material to the ablation element, and a controller coupled to the ablation device to adjust the fluid flow rate or pressure of the fluid flow to the at least one ablation element or the tissue being ablated. In the related methods to ablate these tissues, the invention encompasses the steps of providing an ablation device with at least one ablation element and at least one piezoelectric micropump connecting a source of a flowable material to the ablation device, and controlling an energy source to drive the ablation element and the piezoelectric micropump to ablate tissue and irrigate and/or cool the tissue being ablated.

Any of the devices, systems, or methods for using them, can encompass the use of various energy sources connected to the ablation element or elements and the one or more piezoelectric micropumps. The energy source can be a high frequency energy source, a radio frequency energy source, and a combination of multiple energy sources. For example, an ablation element can be connected to one energy source and a piezoelectric micropump connected to another energy source. Each of these energy sources can be set to operate at different or independent frequencies or frequency ranges. Where a single energy source is used, a first fraction of energy output by the single energy source drives the at least one ablation element and a second fraction of energy output by the single energy source drives the piezoelectric micropump. In one example, a medical device for endocardial or epicardial tissue ablation procedures can comprise a high frequency energy source, at least one ablation element responsive to the high frequency energy source, a piezoelectric micropump, and a passageway to convey a flowable material, preferably physiological solutions or saline solutions, from a piezoelectric micropump to a location proximate to the ablation element.

In addition, any of the ablation devices, systems, or methods of using them, can comprise a controller for automatically controlling the flow of fluid through the one or more piezoelectric micropumps. In advantageous embodiments, the controller or controllers are configured to control a flow rate or the pressure of a flowable material moved by a piezoelectric micropump. As noted herein, various flow rate ranges can be pre-selected for different micropumps and designs therefor.

One advantage of the present invention is that the flow or pressure can also be changed in response to at least one of: tissue temperature; ablation element temperature; the degree of contact between at least one ablation element and the tissue being ablated; and/or the transducer impedance associated with the ablation element used. In this aspect, the invention obviates the need for manual adjustments.

Another advantage of the present invention is more accurate and controlled delivery of fluid to the target tissue during the ablation procedure, resulting in a decreased risk of burning and ensuring adequate contact between the ablation device and the target tissue throughout the procedure.

A further advantage of the present invention is a decrease in the overall size or length of the ablation device and more accurate and controlled fluid flow to the target tissue throughout the procedure for which it is used.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 illustrates an ablation device according to one embodiment of the present invention, incorporating a piezoelectric micropump in a manifold.

FIG. 2A depicts the ablation device of FIG. 1, having multiple piezoelectric micropumps.

FIG. 2B illustrates an enlarged view of the manifold region of the device shown in FIG. 2A.

FIG. 3 provides a schematic diagram of an exemplary piezoelectric micropump.

FIG. 4 depicts another embodiment of an ablation device according to the present invention, having a single piezoelectric micropump.

FIG. 5 illustrates the distal end of the ablation device of FIG. 4.

FIG. 6 depicts the ablation device of FIG. 4, having multiple piezoelectric micropumps.

FIG. 7 illustrates the distal end of the ablation device of FIG. 6.

FIG. 8 schematically illustrates a system for ablating tissue having a single energy source.

FIG. 9 depicts a system for ablating tissue having multiple energy sources.

### DETAILED DESCRIPTION OF THE INVENTION

As used herein, the words “preferred,” “preferentially,” and “preferably” refer to embodiments of the invention that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention and no disclaimer of other embodiments should be inferred from the discussion of a preferred embodiment or a figure showing a preferred embodiment.

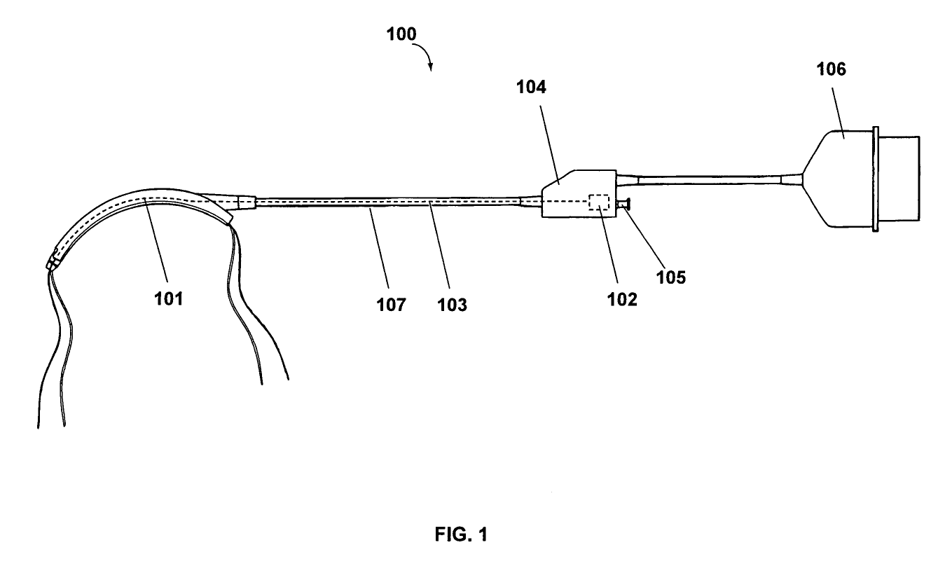

With reference to FIG. 1, an ablation device according to one embodiment of the present invention is shown. The ablation device 100 includes a plurality of ablation elements 101 arranged on a curved surface, a piezoelectric pump 102 within a manifold 104, and a passageway 103 connecting the manifold 104 and piezoelectric pump 102 to the ablation elements 101. Preferably, a piezoelectric pump 102 is integrated into the manifold 104 of the device. A person of skill in the art would appreciate, however, that a piezoelectric pump may be integrated elsewhere, for example, along the passageway 103, or at or near the ablation elements 101. Alternatively, a piezoelectric pump may be coupled to the device, for example, near the manifold 104 via a standard luer connection 105.

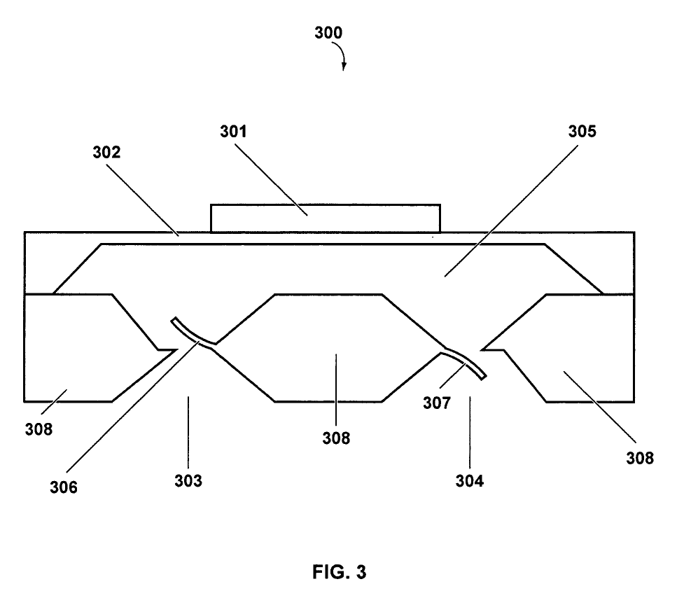

In preferred embodiments, a piezoelectric pump usable according to the present invention is a micropump 300 actuated by a piezoelectric disk 301 (FIG. 3). In general, piezoelectric micropumps of a variety of configurations can be selected for use according to the invention. In the example shown in FIG. 3, a piezoelectric disk 301 is bonded to a membrane or diaphragm 302. The diaphragm 302 contacts or is bonded to a pump body 308, and may overlay a pump chamber 305. A piezoelectric disk 301 is coupled to a power source as described herein or known in the art. As the piezoelectric disk is actuated to a desired vibration frequency, the translated action on fluid in the chamber sounded in part by the membrane is forced in a direction through the pump. The use of multiple chambers, various valves, the geometry of the chamber, the membrane or diaphragm, and the shape of the piezoelectric disk or disks can all or in combination be used to control fluid flow rates and direction through the pump.

The exemplified FIG. 3 pump has an inlet lumen 303 and an outlet lumen 304. The pump may also include an inlet valve 306 and an outlet valve 307. The inlet and outlet valves 306, 307 may be no-moving parts (NVP)-type valves. Alternatively, the pump may have nozzles instead of valves. The pump body 308 is preferably made of hard, biocompatible polymer material, but may be made of any other suitable material.

The piezoelectric disk 301 is preferably made of a piezoelectric ceramic material, such as, for example, lead zirconate titanate (PZT) or barium titanate, but may be made of any other suitable piezoelectric material. The foregoing description and accompanying drawings are illustrative of a type of piezoelectric pump that may be used in accordance with the present invention; however a person of skill in the art will appreciate that appropriate piezoelectric micropumps for use in the invention may be modified from those specifically depicted here, selected from one or more of those available to one skilled in the art, and used without departing from the spirit and scope of the instant invention.

Referring again to FIG. 1, the passageway 103 is preferably an elongated tube having a lumen for carrying a flowable material, such as saline or hypertonic saline; however the passageway 103 may have any suitable shape. The passageway may also be enclosed in a casing 107. The passageway 103 is preferably made of a biologically acceptable polymeric material, such as silicone, urethane, or polyvinyl chloride (PVC). One of ordinary skill will appreciate, however, that any suitable materials and methods may be used to form the passageway without departing from the spirit and scope of the present invention.

The device 100 has at least one ablation element, preferably about 5 to about 30 ablation elements, more preferably about 10 to about 25 ablation elements, and most preferably less than about 15 elements. Ablation devices that are suitable for the present invention are described in U.S. Pat. No. 7,052,493, which is incorporated herein by reference in its entirety. It should be understood, however, that any number of ablation elements may be used depending upon the specific application for the ablation device. For example, the ablation device may be used to extend around only a single vessel, such as the aorta, a pulmonary vein, the superior vena cava, or inferior vena cava, in which case the ablation device preferably includes about 4 to about 12 ablation elements, and more preferably includes about 8 ablation elements.

The ablation elements 101 may comprise any element for directing and delivering ablating energy to the cardiac tissue, including, but not limited to a radiofrequency electrode, a microwave transmitter, a cryogenic element, a laser or an ultrasonic transducer. The ablation elements preferably have a width of about 1 mm to about 15 mm, and more preferably of about 10 mm, and a length of about 2 mm to about 25 mm, and more preferably of about 12 mm.

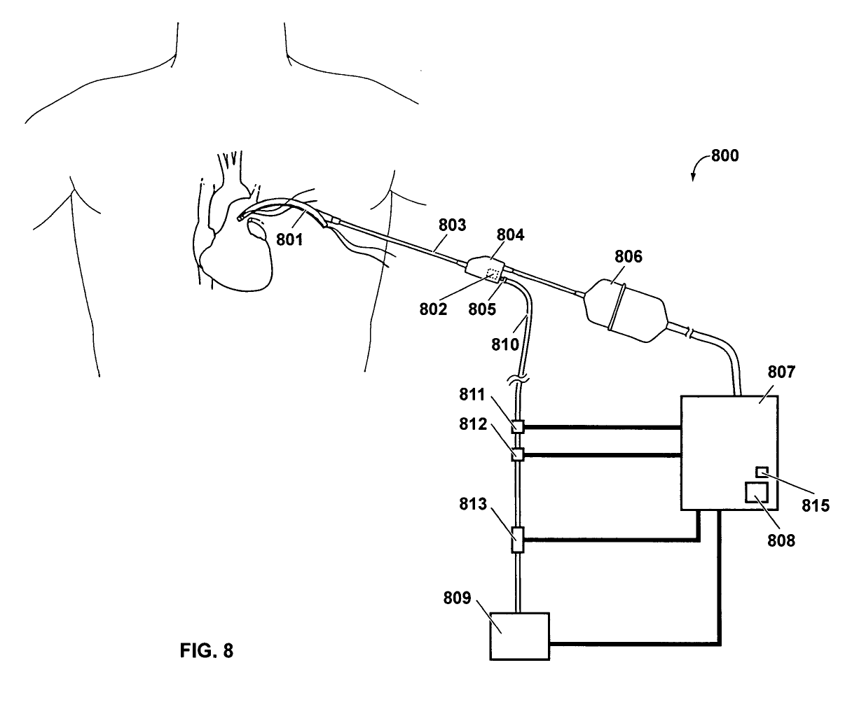

The device 100 may also include a plug 106 usable to couple the ablation device to a controller 807 (FIG. 8). The device 100 may further include one or more energy sources coupled to the ablation elements 101 and the piezoelectric pump 102. The one or more energy sources may be part of the controller or separate therefrom. The one or more energy sources may include a high frequency energy source, an RF energy source, any combination of the two or any other suitable energy source or combination of energy sources. The ablation device 100 may further include a splitter (not shown) for directing a first percentage of input energy to the one or more ablation elements 101 and a second percentage of input energy to the piezoelectric pump 102.

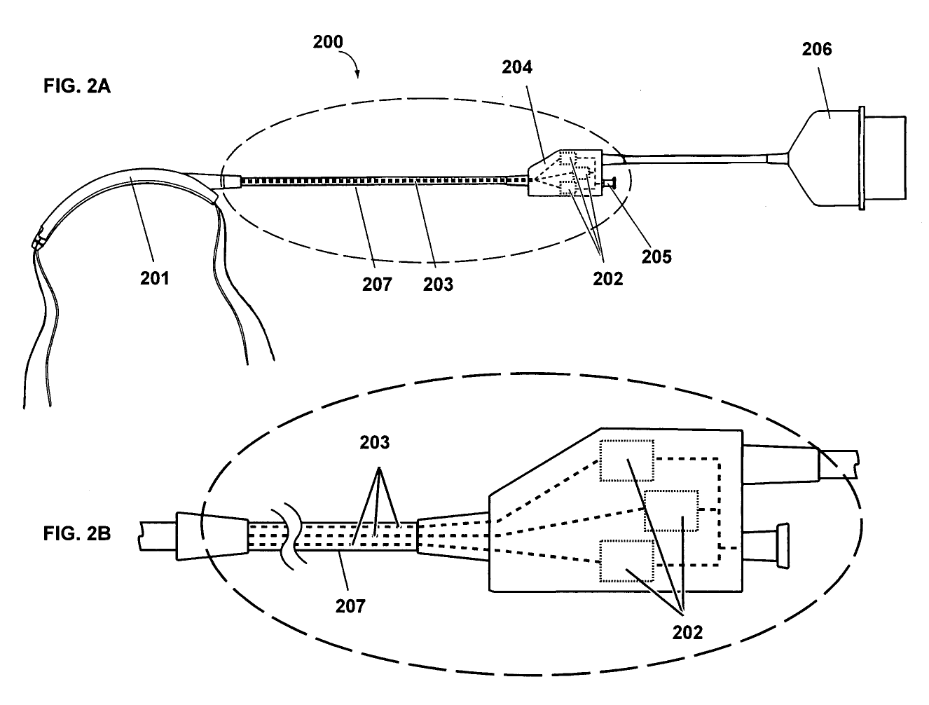

With reference to FIGS. 2A-2B, another ablation device according to the present invention is shown. The ablation device 200 includes a plurality of ablation elements 201 arranged on a curved surface, two or more piezoelectric pumps 202 within a manifold 204, and two or more passageways 203 connecting the piezoelectric pumps 202 to the ablation elements 201. The two or more passageways 203 may be enclosed within a casing 207. Preferably, each piezoelectric pump 202 is connected to a single ablation element 201, however each piezoelectric pump may be connected to more than one ablation element. For example, each piezoelectric pump may be connected to two ablation elements, three ablation elements, or four or more ablation elements.

In alternate embodiments, two or more piezoelectric pumps 202 may be integrated along two or more passageways 203 at or near the ablation elements 201, or the two or more piezoelectric pumps 202 may be coupled to the device, for example, near the manifold 204 via a standard luer connection 205.

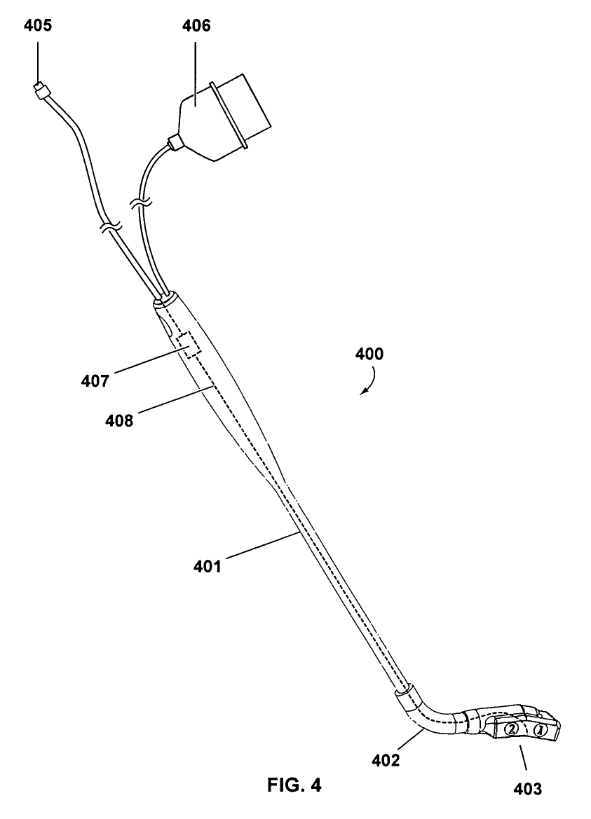

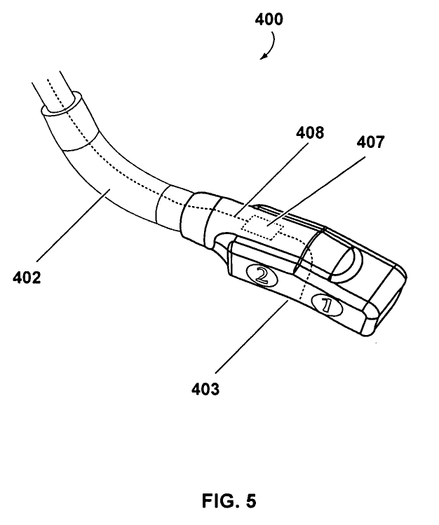

Referring now to FIGS. 4 and 5, another medical device for ablating tissue is shown. The device 400 has a shaft 401, which is relatively rigid, with a flexible distal portion 402. The distal portion of the shaft can be shaped by a user (i.e., a physician) into a variety of positions to accommodate the angle of introduction of the ablating elements 403 into the patient and the target surface orientation. The distal portion may include a stacked coil contained within a sheath that can be deformed by the user and retain the deformed shape.

In a particularly preferred device as shown in FIG. 4, the device 400 has at least one ablation element, and preferably two ablation elements 403. The ablation device may, of course, have more or less than two ablation elements. The ablation elements may be any suitable ablation elements, such as focused ultrasound elements, radio frequency (RF) elements, laser elements, and microwave elements. The ablation elements may be fixed relative to one another, or, alternatively, may have a flexible or malleable connection therebetween in order to adjust the relative orientation or position of ablation elements.

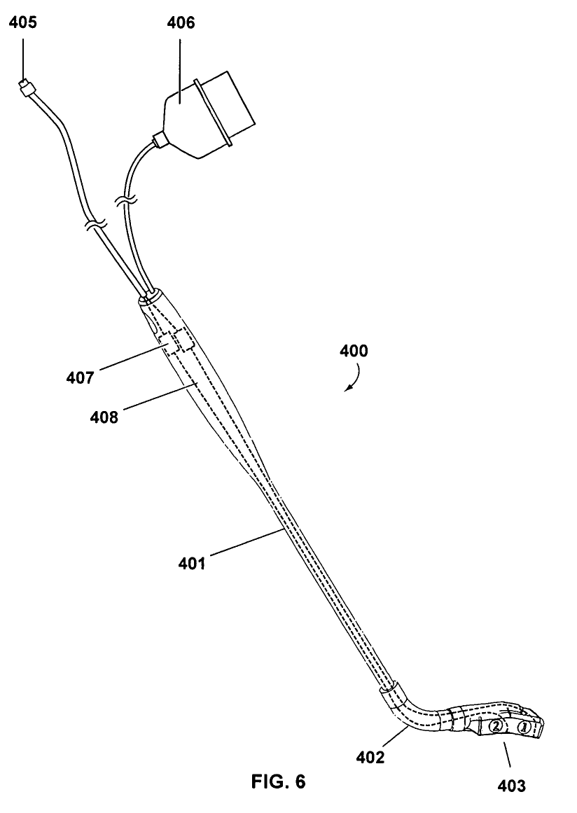

The device 400 also includes a piezoelectric pump 407 and a passageway 408 connecting the piezoelectric pump 407 and the ablation elements 403. The passageway 408 extends through the shaft 401 to the ablation elements 403. The piezoelectric pump 407 is preferably integrated near the proximal end of the shaft 401, however the piezoelectric pump may alternatively be integrated at the distal end of the passageway adjacent the ablation elements 403 (FIG. 5). Alternatively, the piezoelectric pump may be connected to the device at its proximal end, for example, via a standard luer connection 405. A standard luer connection 405 may also be used to couple the ablation device to a source of a flowable material. The ablation device may further include a plug 406 usable to couple the ablation device to a controller 807 (FIG. 6) for controlling the device.

The device 400 may also include one or more energy sources coupled to the ablation elements 403 and the piezoelectric pump 407. The one or more energy sources may be part of the controller 807 or separate therefrom. The one or more energy sources may include a high frequency energy source, an RF energy source, any combination of the two or any other suitable energy source or combination of energy sources. The ablation device 400 may further include a splitter (not shown) for directing a first percentage of input energy to the one or more ablation elements 403 and a second percentage of input energy to the piezoelectric pump 407.

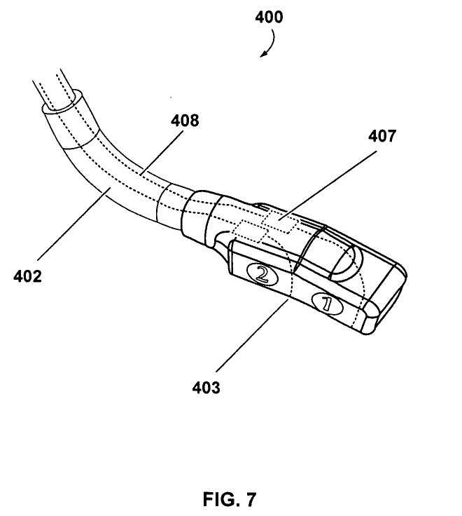

In another embodiment, the device 400 may have two piezoelectric pumps 407 connected to the ablation elements 403 (FIG. 6). The two piezoelectric pumps may be connected to the ablation elements via a single passageway, or each piezoelectric pump 407 may be connected to the ablation elements via its own passageway 408. The piezoelectric pumps may be integrated into the device at the proximal end of the shaft (FIG. 6), or the piezoelectric pumps may be integrated at the distal end of the device near the ablation elements 403 (FIG. 7).

Referring now to FIG. 8, an ablation system according to one embodiment of the present invention is shown. The ablation system 800 includes a controller 807 that operates in any of he modes described herein, and preferably operates to deliver high frequency energy. The ablation system 800 may be used to wrap an ablation device, such as any ablation device described herein (e.g., the ablation devices illustrated in FIGS. 1 and 4) or another suitable device, around the pulmonary veins at an epicardial location. The ablation system 800 further includes a source of a flowable material 809, such as saline, which may be driven by a piezoelectric micropump 802 to deliver the flowable material to the tissue via a passageway 803. The source of flowable material 809 may be connected to a manifold 804 via a standard luer connector 805.

The ablation system 800 may additionally include at least one energy source 808, which may be integrated into the controller, or may be a separate therefrom. For example, the ablation system may include a single energy source 808 that delivers energy to both the ablation elements 801 and the piezoelectric pump 802. The energy source may be a high frequency or RF energy source, or any other suitable energy source. The ablation system may further include a splitter 815 for directing a first percentage of input energy to the one or more ablation elements 801 and a second percentage of input energy to the piezoelectric micropump 802.

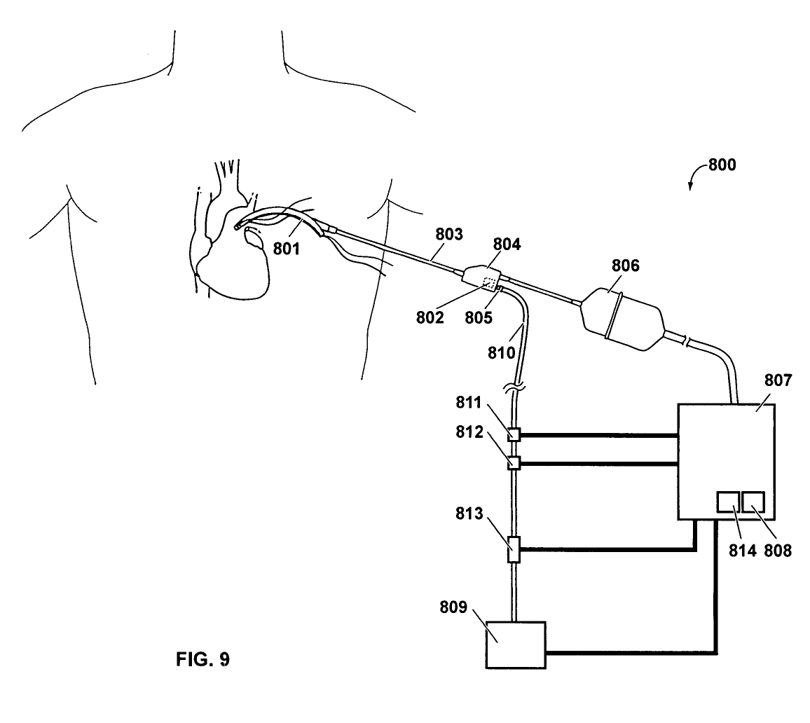

In an alternative embodiment shown in FIG. 9, the ablation system may include two or more energy sources either integrated into the controller 807 separate therefrom. For example, a first energy source 808 may drive the ablation elements 801 and a second energy source 814 may drive the piezoelectric pump 802. The first and second energy sources may deliver the same type of energy, such as, for example, high frequency energy, or the two energy sources may deliver different types of energy such as, for example, high frequency and RF energy. One skilled in the art would appreciate that different types and combinations of energy sources can be used without departing from the spirit and scope of the present invention.

The piezoelectric pump 802 preferably operates at a lower frequency than the ablation elements, but it may operate at the same or a higher frequency than the ablation elements. In any of the embodiments of the invention designed to be used in a cardiac or atrial ablation method, as shown in the exemplary High Intensity Focused Ultrasound ablation system of FIGS. 8 and 9, the piezoelectric pump using saline fluid is preferably set to an average flow rate of at least 1.0 cc/sec to each cell, although lower or higher flows may be used. The fluid may be cooled, or even heated, by passing the fluid through a heat exchanger 813 or controlling the initial temperature of the fluid. The fluid is preferably delivered at a temperature of no more than 40 degrees C., and more preferably no more than 25 degrees C. to cool the tissue and/or ablating element during use. Depending on the piezoelectric pump and design selected, the frequency of vibration of piezoelectric elements or disks can be selected to achieve the desired fluid flow rates.

Although it is preferred to vary the frequency of the energy delivered to the ablating elements when ablating the tissue, the ablation elements may, of course, be operated at a single frequency. Various treatment methods for delivering energy to the ablation elements are described in U.S. Pat. No. 7,052,493. In a first treatment method, the ablation elements are activated at a frequency of about 2 MHz to about 7 MHz, and preferably of about 3.5 MHz, and a power of about 80 watts to about 150 watts, and preferably of about 130 watts, in short bursts. Following treatment at the first frequency, the ablation elements are preferably operated at a frequency of about 2 MHz to about 14 MHz, more preferably about 3 MHz to about 7 MHz, and most preferably about 6 MHz, and a power of about 20 watts to about 80 watts, and preferably about 60 watts. As a final treatment, the ablation elements are preferably operated at a frequency of at least about 3 MHz and about 16 MHz, and preferably at about 6 MHz. In a preferred method, the ablation elements are operated at about 2 watts to about 20 watts, and more preferably about 15 watts.

The ablation devices and systems of the present invention may also include one or more temperature sensors (not shown), preferably thermocouples, positioned near the ablation elements in order to measure temperature. The temperature sensors may be coupled to the controller 807, for example via the plug 806, for monitoring purposes and to provide temperature feedback for controlling the ablation process. The devices and systems of the present invention may further include one or more flow rate sensors 811 and/or pressure sensors 812 positioned, for example, along the fluid inlet lumen 810 or near the ablation elements 801. The pressure and/or flow rate sensors may also be coupled to the controller 807. The pressure and/or flow rate sensors determine when the ablation elements are adequately secured to the tissue. If the degree of contact is not adequate, the pressure and/or flow rate will be higher than expected. The flow rate sensors can also be used to measure fluid flow into and out of the device to determine whether fluid is leaking from the ablation elements, which also indicates a poor seal. Air bubbles or gaps between the ablation elements and the target tissue can inhibit energy transfer and, in particular, can reduce the efficiency of high frequency ultrasound and RF energy transfer. The degree of contact between an ablation element and the tissue can also be measured in other ways, for example, using a force electrode or a pacing electrode. Alternatively, a pressure sensitive conductive composite (PSCC) may be used to assess contact between an ablation element and a tissue. The electrical resistance of a PSCC varies inversely in proportion to the pressure that is applied to the PSCC.

Preferably, the ablation at each ablation element is controlled based on temperature measured at the temperature sensors. The controller 807 is programmed to automatically adjust the flow rate of the flowable material in response to temperature changes. For example, as the temperature of a tissue or an ablation element increases, the controller will increase the flow rate of the flowable material to cool the tissue. Alternatively, as the temperature decreases, the controller will decrease the flow rate of the flowable material. For example, the controller may be configured to maintain a near surface (NS) temperature of about 0 degrees C. to about 80 degrees C., more preferably about 20 degrees C. to about 80 degrees C., and most preferably about 40 degrees C. to about 80 degrees C. The controller may also incorporate a multiplexer that delivers ablating energy only to those ablation elements having a temperature below a threshold temperature. Alternatively, the multiplexer may deliver ablating energy only to those ablation elements registering the coolest temperatures.

In the system of FIG. 8, the flowable material is preferably supplied at an average flow rate of at least about 0.24 cc/sec, more preferably at least about 0.50 cc/sec, and most preferably at least about 1.0 cc/sec to each ablation element cell, although lower or higher flow rates may be used. The flowable material is preferably delivered to the ablation elements at a set pressure that results in the desired average flow rate through the ablation elements. The flowable material is preferably delivered at a pressure of 0.5 psi to 2.0 psi. The flowable material may be heated or cooled as desired or required by passing it through a heat exchanger prior to delivery to the ablation elements. The flowable material is preferably delivered at a temperature of no more than about 40 degrees C., and more preferably at a temperature of no more than about 20 degrees C., to cool the tissue and/or the ablation element.

The controller 807 may also be programmed to automatically adjust the flow rate of the flowable material in response to changes in transducer impedance and in the degree of contact between the ablation elements and the tissue. For example, as the electrical impedance of the transducer increases, the controller will increase the flow rate of the flowable material and as the electrical impedance of the transducer decreases the controller will decrease the flow rate of the flowable material. Further, as the degree of contact between the ablation elements and the tissue decreases, indicating a poor seal, the controller will increase the flow rate of the flowable material. Alternatively, as the degree of contact between the ablation elements and the tissue increases, indicating a good seal, the controller will decrease the flow rate of the flowable material.

A method of ablating tissue is now described. A source of flowable material is connected to an ablation device having at least one ablation element and a piezoelectric pump, such as, for example, the devices described herein with reference to FIGS. 1 and 4, or any other suitable ablation device. The flowable material may be saline, hypertonic saline, or any other suitable fluid. The flowable material is delivered to a location proximate the ablation element via a passageway connecting the piezoelectric pump and the ablation element. At least one energy source is used to drive the at least one ablation element and the piezoelectric pump. The energy source may be a high frequency energy source, an RF energy source, any combination of the two or any other suitable energy source or combination of energy sources. When a single energy source is used the energy may be split using a splitter such that a first fraction of energy output drives the ablation element and a second fraction of energy output drives the piezoelectric pump.

A controller be connected to the ablation device to control the flow of the flowable material. The controller is configured to monitor at least one of tissue temperature and degree of contact between the ablation element and the tissue. The controller is further configured to adjust a flow rate of the flowable material in response to at least one of changes in tissue temperature, ablation element temperature, transducer impedance and changes in degree of contact between the ablation element and the tissue. For example, when the tissue or ablation element temperature increases, the controller increases the flow rate of the flowable material and when the temperature decreases, the controller decreases the flow rate of the flowable material. When the degree of contact between the ablation element and the tissue decreases, the controller increases the flow rate of the flowable material to provide a better seal between the ablation element and the tissue.

Although several embodiments of this invention have been described above with a certain degree of particularity, those skilled in the art could make numerous alterations to the disclosed embodiments without departing from the spirit or scope of this invention. All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting. Changes in detail or structure may be made without departing from the spirit of the invention as defined in the appended claims.

What is claimed is:

1. A device for ablating cardiac tissue, comprising:
at least one ablation element;
at least one piezoelectric micropump; and
a passageway to convey a flowable material between said piezoelectric micropump and a location proximate said at least one ablation element.

2. The device of claim 1, having a plurality of ablation elements.

3. The device of claim 1, wherein said at least one ablation element is a high intensity focused ultrasound cell or element.

4. The device of claim 3, wherein multiple cells or elements are used and each cell or element is connected to a passageway from a piezoelectric micropump.

5. The device of claim 4, wherein the flow rate of the flowable material to each cell or element is about 1 cc/sec or more.

6. The device of claim 1, having a single piezoelectric micropump.

7. The device of claim 1, having a plurality of piezoelectric micropumps.

8. The device of claim 1, further comprising at least one energy source coupled to said at least one ablation element and said piezoelectric micropump.

9. The device of claim 8, wherein a single energy source is coupled to both said at least one ablation element and said piezoelectric micropump.

10. The device of claim 1, further comprising a first energy source coupled to said at least one ablation element and a second energy source coupled to said piezoelectric micropump.

11. The device of claim 1, further comprising a splitter, wherein said splitter directs a first percentage of input energy to said at least one ablation element and a second percentage of input energy to said piezoelectric micropump.

12. The device of claim 1, wherein said at least one ablation element operates at a first frequency and wherein said piezoelectric micropump operates at a second frequency, said second frequency being lower than said first frequency.

13. The device of claim 1, further comprising a controller configured to control a flow rate or pressure of a flowable material moved by said piezoelectric micropump in response to at least one of tissue temperature, ablation element temperature, degree of contact between said at least one ablation element and the tissue, and transducer impedance.

14. The device of claim 13, wherein said controller is configured to increase the flow rate of the flowable material when the tissue temperature increases, and to decrease the flow rate of the flowable material when the tissue temperature decreases.

15. The device of claim 13, wherein said controller is configured to decrease the flow rate of the flowable material when the degree of contact between said at least one ablation element and the tissue increases, and to increase the flow rate of the flowable material when the degree of contact between said at least one ablation element and the tissue decreases.

16. The device of claim 13, wherein said controller is configured to increase the flow rate of the flowable material when the ablation element temperature increases, and to decrease the flow rate of the flowable material when the ablation element temperature decreases.

17. The device of claim 13, wherein said controller is configured to increase the flow rate of the flowable material when the transducer impedance increases, and to decrease the flow rate of the flowable material when the transducer impedance decreases.

18. The device of claim 1, further comprising a manifold located remotely from said at least one ablation element, wherein said manifold houses said at least one piezoelectric micropump.

19. An irrigated medical device comprising:
at least one high intensity focused ultrasound element; and
at least one micropump, said micropump having a piezoelectric material; a fluid chamber; a membrane; and a conduit to permit flow of a fluid into and out of said chamber, whereby said micropump is capable of being used inside the body during a surgical procedure.

20. The device of claim 19, having a plurality of micropumps.

21. The device of claim 19, having a plurality of high intensity focused ultrasound elements.

22. A system for ablating cardiac tissue, comprising:
an ablation device having at least one ablation element, at least one piezoelectric micropump and a passageway to convey a flowable material between said piezoelectric micropump and a location proximate said at least one ablation element; and
a controller coupled to said ablation device, the controller being programmed to monitor at least one of tissue temperature, ablation element temperature, transducer impedance degree of contact between said at least one ablation element and the tissue, wherein said controller automatically adjusts one or more of a flow rate of the flowable material and a pressure of the flowable material in response to one or more of temperature changes, changes in the degree of contact between said at least one ablation element and the tissue, and changes in transducer impedance.

23. The system of claim 22, wherein said at least one ablation element is a high intensity focused ultrasound element.

24. The system of claim 22, having a plurality of ablation elements.

25. The system of claim 22, having a plurality of piezoelectric micropumps.

26. The system of claim 22, further comprising a first energy source coupled to said at least one ablation element and a second energy source coupled to said at least one piezoelectric micropump.

27. The system of claim 22, wherein said controller is configured to increase the flow rate of the flowable material when the tissue temperature increases, and to decrease the flow rate of the flowable material when the tissue temperature decreases.

28. The system of claim 22, wherein said controller is configured to decrease the flow rate of the flowable material when the degree of contact between said at least one ablation element and the tissue increases, and to increase the flow rate of the flowable material when the degree of contact between said at least one ablation element and the tissue decreases.

29. The system of claim 22, wherein said controller is configured to increase the flow rate of the flowable material when the ablation element temperature increases, and to decrease the flow rate of the flowable material when the ablation element temperature decreases.

30. The system of claim 22, wherein said controller is configured to increase the flow rate of the flowable material when the transducer impedance increases, and to decrease the flow rate of the flowable material when the transducer impedance decreases.

31. A method of ablating tissue, comprising:
providing an ablation device having at least one ablation element and at least one piezoelectric micropump;
connecting a source of a flowable material to said ablation device such that said at least one piezoelectric micropump may deliver the flowable material from said source of flowable material to a location proximate said at least one ablation element; and
utilizing at least one energy source to drive said at least one ablation element and said at least one piezoelectric micropump, whereby tissue is ablated and flowable material is directed toward the tissue.

32. The method of claim 31, further comprising connecting a controller to said ablation device, wherein said controller is configured to monitor at least one of tissue temperature, ablation element temperature, degree of contact between said at least one ablation element and the tissue, and transducer impedance and to adjust a flow rate of the flowable material in response to at least one of temperature changes, changes in the degree of contact between said at least one ablation element and the tissue, and changes in transducer impedance.

33. The method of claim 32, wherein the flow rate of the flowable material increases when the tissue temperature increases and decreases when the tissue temperature decreases.

34. The method of claim 32, wherein the flow rate of the flowable material increases when the ablation element temperature increases and decreases when the ablation element temperature decreases.

35. The method of claim 32, wherein the flow rate of the flowable material increases when the degree of contact between said at least one ablation element and the tissue decreases and decreases when the degree of contact between said at least one ablation element and the tissue increases.

36. The method of claim 32, wherein the flow rate of the flowable material increases when the transducer impedance increases and decreases when the transducer impedance decreases.

37. The method of claim 31, wherein the step of utilizing at least one energy source to drive the at least one ablation element and the piezoelectric micropump comprises utilizing a first energy source to drive said at least one ablation element and a second energy source to drive said piezoelectric micropump.
